(19) United States
(12) Patent Application Publication
(10) Pub. No.: US 2005/0070984 A1
(43) Pub. Date: Mar. 31, 2005

(54) Extendable and retractable lead with an active fixation assembly

(76) Inventor: Gregory L. Sundberg, Stillwater, MN (US)

Correspondence Address: Schwegman, Lundberg, Woessner & Kluth, P.A., P.O. Box 2938, Minneapolis, MN, 55402, US

(21) Appl. No.: 10/673,926

(22) Filed: Sep. 29, 2003

(51) Int. Cl. A61N 1/05

(52) U.S. Cl. 607/122

(57) ABSTRACT

An extendable and retractable lead includes a lead body which extends from a distal end to a proximal end, where a conductor is disposed within the lead body. The lead assembly further includes a piston that is movably disposed within the lead body. A fixation helix is supported by the piston at a first portion of the fixation helix, where the first portion of the fixation helix forms a drive mechanism that advances and/or retracts the fixation helix.

### TECHNICAL FIELD

The present disclosure relates generally to implantable leads. More particularly, it pertains to leads having an extendable and retractable fixation mechanism.

### TECHNICAL BACKGROUND

Electrodes have been used to stimulate contraction of the heart or to reverse certain life threatening arrhythmias, where electrical energy is applied to the heart via the electrodes to return the heart to normal rhythm. Electrodes have also been used to sense and deliver pacing pulses to the atrium and ventricle. Cardiac pacing may be performed by a transvenous method or by electrodes implanted directly onto the epicardium. For transvenous pacing systems, a lead having an electrode is positioned in the right ventricle and/or in the right atrium through a subclavian vein, and the proximal electrode terminals are attached to a pacemaker which is implanted subcutaneously.

Some lead designs have “floating” electrodes or electrodes which are not attached to the endocardial wall of the heart. The floating electrodes lay in the blood pool or against the endocardial wall of the heart and the electrode may move slightly within the heart. As an alternative to floating electrodes, leads have been provided with passive fixation elements that affix the electrode to the endocardial wall over time. Another alternative to floating electrodes is active fixation element.

Active fixation elements, such as a helix, have also been provided with distal ends of leads which allow a lead to be affixed to the endocardial wall. The helix is rotated to screw the lead into the endocardial wall. However, as lead designs become smaller, it can be difficult to provide robust, cost effective designs that allow for a helix to be advanced out of and retracted in to a lead body.

Accordingly, what is needed is an extendable and retractable helix mechanism that addresses the above concerns.

### SUMMARY

An extendable and retractable lead includes a lead body which extends from a distal end to a proximal end, where a conductor is disposed within the lead body. The lead assembly further includes a piston that is movably disposed within the lead body. A fixation helix is supported by the piston at a first portion of the fixation helix, where the first portion of the fixation helix forms a drive mechanism that advances the fixation helix.

Several options for the lead assembly are as follows. For example, in one option, the first portion of the fixation helix is coupled with the piston. In another option, the piston has a recess therein, and at least a portion of the first portion of the fixation helix is disposed within the recess, and the recess optionally has a helical shape. In one option, the recess has a first width and the first width is less than a diameter of the first portion of the fixation helix. In another option, the fixation helix has an inner diameter and the piston has an outer diameter, and the outer diameter is greater than the inner diameter prior to coupling the fixation helix with the piston.

The lead assembly also, in one option, includes a housing that includes a guide is disposed on an inner surface of the housing portion, and the guide guides the drive mechanism of the fixation helix. The guide, in one option, is a helical guide or a segmented helical guide.

A method is also provided and includes providing a lead assembly that includes a lead body extending from a distal end to a proximal end, a conductor disposed within the lead body, a piston movably disposed within the lead body, and a fixation helix supported by the piston at a first portion of the fixation helix, where the first portion of the fixation helix forms a drive mechanism. The method further includes rotating the fixation helix, and longitudinally driving the fixation helix with the drive mechanism.

Several options for the method are as follows. For example, in one option, the method includes recessing at least a part of the first portion of the helix within the piston, such as recessing approximately ⅓ to ½ of a diameter of the fixation helix within the piston. In another option, the method further includes recessing at least a part of the first portion of the helix within a helical groove of the piston. In one option, the method further includes coupling the first portion of the fixation helix with the piston.

These and other embodiments, aspects, advantages, and features of the present disclosure will be set forth in part in the description which follows, and in part will become apparent to those skilled in the art by reference to the following description and referenced drawings or by practice thereof. The aspects, advantages, and features of the invention are realized and attained by means of the instrumentalities, procedures, and combinations particularly pointed out in the appended claims and their equivalents.

### BRIEF DESCRIPTION OF THE DRAWINGS

FIG. 1 is a diagram illustrating a system for delivering and/or receiving signals to and from the heart constructed in accordance with one embodiment.

FIG. 2 is a cross-section illustrating a portion of a lead constructed in accordance with one embodiment.

FIG. 3 is a cross-section illustrating a distal end of a lead constructed in accordance with one embodiment.

FIG. 4 is a cross-section illustrating a distal end of a lead constructed in accordance with another embodiment.

FIG. 5A is a cross-section illustrating a housing portion constructed in accordance with one embodiment.

FIG. 5B is a cross-section illustrating a housing portion constructed in accordance with one embodiment.

FIG. 5C is a detail of a portion of the housing taken from FIG. 5B.

### DESCRIPTION OF THE EMBODIMENTS

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Therefore, the following detailed description is not to be taken in a limiting sense, and the scope of the present invention is defined by the appended claims and their equivalents.

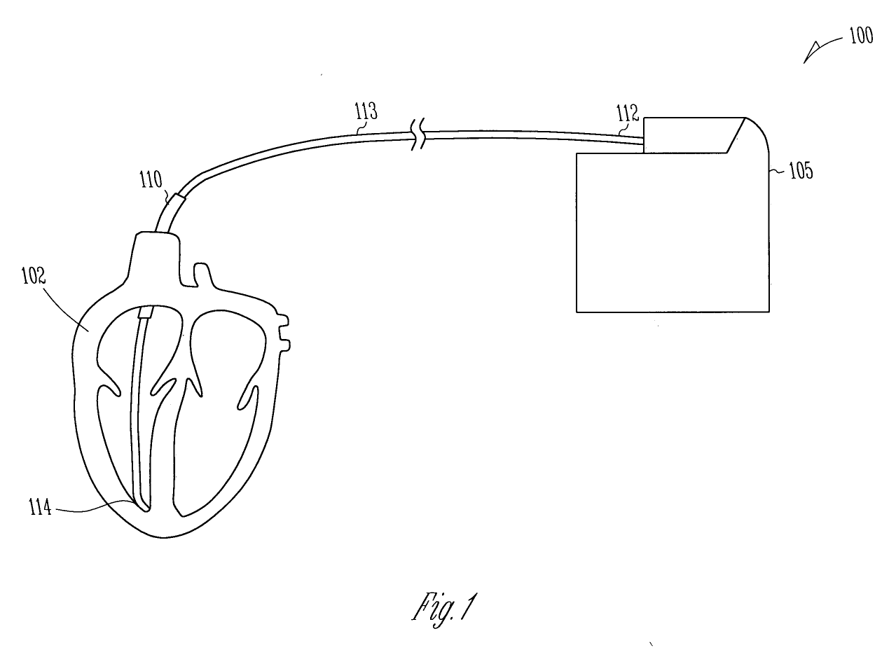

An extendable and retractable lead 110 and lead system 100 are illustrated in FIG. 1. FIG. 1 is a diagram of a system 100 for delivering and/or receiving electrical pulses or signals to stimulate and/or sense the heart 102. The system 100 includes a pulse generator 105 and a lead 110. The pulse generator 105 includes a source of power as well as an electronic circuitry portion. The pulse generator 105, in one option, is a battery-powered device which generates a series of timed electrical discharges or pulses. The pulse generator 105 is generally implanted into a subcutaneous pocket made in the wall of the chest. Alternatively, the pulse generator 105 is placed in a subcutaneous pocket made in the abdomen, or in other locations.

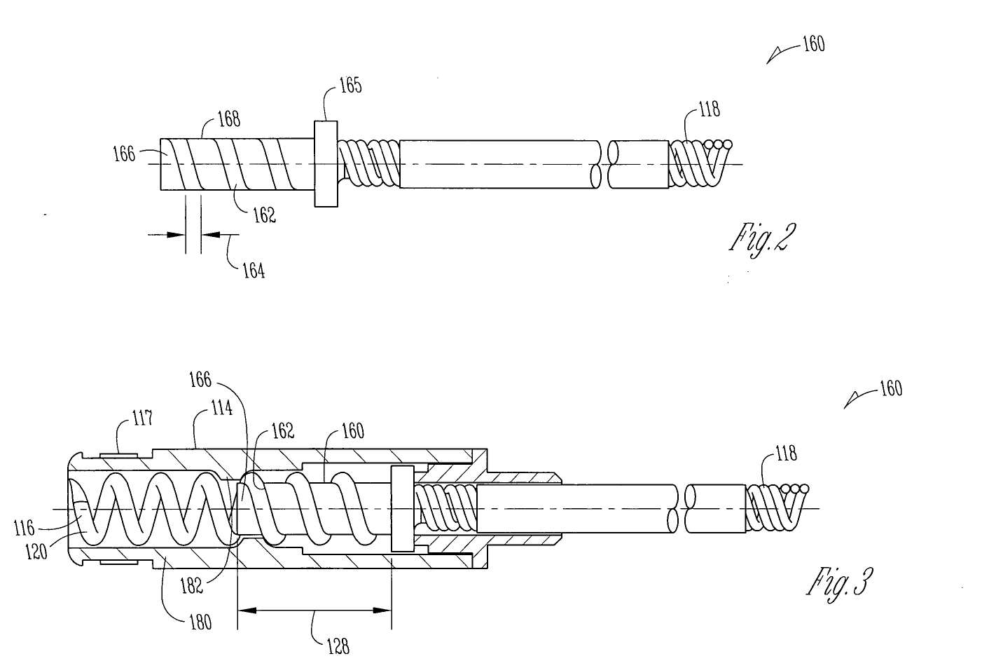

The lead 110 includes a lead body 113 which extends from a proximal end 112, where it is coupled with the pulse generator 105, as further discussed below. The lead 110 extends to a distal end 114, which is coupled with a portion of a heart 102, when implanted. The distal end 114 of the lead 110 includes at least one electrode 116 (FIG. 3) which electrically couples the lead 110 with the heart 102. At least one electrical conductor 118 (FIGS. 2 and 3) is disposed within the lead 110 and extends, in one option, from the proximal end 112 to the distal end 114 of the lead 110. The at least one electrical conductor 118 electrically couples the electrode 116 with the proximal end 112 of the lead 110. The electrical conductors carry electrical current and pulses between the pulse generator 105 and the electrode 116 (FIG. 3).

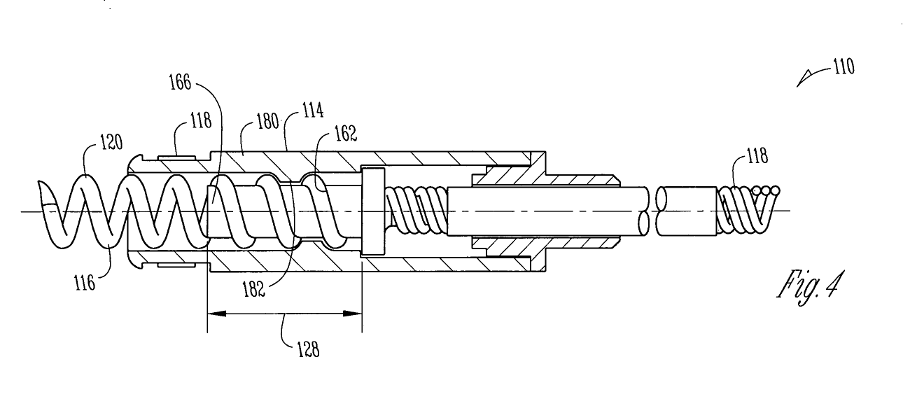

The lead 110 further includes a distal end assembly including a fixation helix 120 (FIGS. 3 and 4), a piston 160 (FIG. 2), and an end housing 180 (FIGS. 3 and 4). FIG. 2 illustrates the piston 160 in greater detail. The piston 160, in one option, is electrically conductive, and allows for the fixation helix 120 (FIG. 3) to be advanced longitudinally through the lead body 113 (FIG. 1). The piston 160 is defined in part by a piston outer diameter 168, and the piston outer diameter 168 is optionally greater than an inner diameter of the fixation helix 120 (FIG. 3), where the dimensions are preformed, for example before the parts are assembled to one another.

The piston 160, in one option, includes features that allow for the fixation helix 120 (FIGS. 3 and 4) to be mechanically and/or electrically coupled with the piston 160. For example, in one option, the piston 160 includes a recess 162 therein. The recess 162 is defined in part by its width 164. The recess 162, that is in one option a groove, extends along a longitudinal portion of the piston 160, and in one option extends near the distal end 166 (FIGS. 2 and 3) of the piston. In one option, the recess 162 wraps around the exterior portion, for example, an outer perimeter of the piston 160. The recess 162, in one option, has a helical shape as it wraps around the piston 160. In another option, the cross-section of the recess 162 has a semi-circular shape. The piston 160 further optionally includes features that prevent over extension of the fixation helix 120 (FIG. 3) from the lead body 113 (FIG. 1). For example, in one option, the piston 160 includes a stop 165 that prevents a physician from over extending the fixation helix from the lead body 113 (FIG. 1).

FIGS. 3 and 4 illustrate one embodiment of the distal end 114 of the lead 110 in greater detail, where FIG. 3 illustrates the lead 110 in a retracted position and FIG. 4 illustrates the lead 110 in an extended position. The distal end 114 includes a housing 180 that houses the fixation helix 120 and piston 160 therein.

The fixation helix 120 is supported by the piston 160 along a first portion 128 of the fixation helix 120. The support of the piston 160 provides mechanical support to the first portion 128 and assists in preventing the first portion 128 of the fixation helix 120 from becoming bent, stretched, or compressed during the longitudinal advancement of the fixation helix 120. In one option, the first portion 128 is at an intermediate portion of the fixation helix 120, and is not at the distal tip of the fixation helix 120 or manipulation of the lead. For example if a physician pulls on the lead after the fixation helix engages tissue. The first portion 128 of the fixation helix 120 forms a drive mechanism for the fixation helix 120, and allows for the fixation helix 120 to advance out of and retract in to the housing 180. In one option, the fixation helix 120 is electrically coupled with the piston 160 and the conductor, and the fixation helix 120 is electrically active.

In one option, the fixation helix 120 is mechanically coupled to the piston 160 along the first portion 128. For example, the fixation helix 120 is partially recessed within the piston 160. In one option, the fixation helix 120 is disposed within a recess 162 of the piston 160, such as a helical groove where part of the helix is buried within the piston 160. The recess 162 optionally has a width that is less than the diameter of the helix 120. In another option, approximately ⅓ of the diameter of the helix 120 is disposed within the recess 162 of the piston 160, along the first portion 128 of the fixation helix 120. In yet another option, about ½ of the diameter of the helix 120 is disposed within the recess 162 of the piston 160, along the first portion 128 of the fixation helix 120. The recess provides support for the helix 120 and assists in preventing stretching and/or compression of the helix during manipulation of the lead within the patient. It should be noted that the fixation helix 120 can be coupled with the piston 160 in other manners or at other locations.

As discussed above, the lead 110 includes the housing 180. In one option the housing is a molded component, with molded features therein. The features that can be molded on or in the housing 180, for example the guide (discussed below), allow for the lead to be made with less expensive components. The molding also allows for repeatable dimension control, including the position of molded inserts, such as a fluoromarker 118. It further allows for the housing 180 and the lead 110 to be made smaller, resulting in less trauma to the patient. The molding process also allows for a greater selection of materials that can be used for the housing 180.

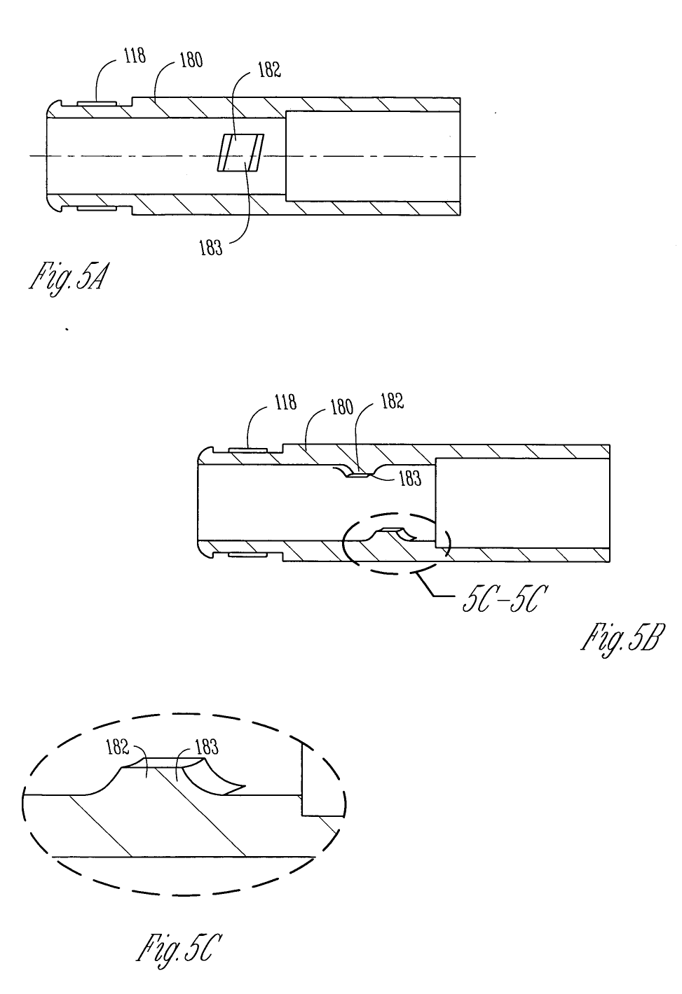

The housing 180 optionally includes a guide 182 therein. The guide 182 extends from an inner surface 184 of the housing 180 and interacts with the first portion 128 of the fixation helix 120. For example, the first portion 128 of the fixation helix 120 rides along the guide 182 to drive the fixation helix 120 longitudinally. In one option, the guide 182 has a helical shape, and optionally wraps around the inner surface 184 of the housing 180. In another option, the guide 182 has a general helical shape, but does not continuously wrap within the housing 180. Instead, the guide 182 has a segmented helical shape, where segments 183 of a helical shape are disposed within the housing 180, as illustrated in FIGS. 5A, 5B, and 5C. This allows for a balanced force load without creating significant amounts of drag between the fixation helix 120 (FIG. 4) and the guide 182. In yet another option, the guide 182 has a rounded cross-sectional shape.

Referring again to FIGS. 3 and 4, during use of the device, the piston 160 is rotated, for example, by rotating the conductor, or terminal pin, or by using a stylet to rotate the piston 160. As the piston 160 rotates, the first portion 128 of the helix 120 that is supported by, and optionally coupled with, the piston 160 rides along the guide 182 and longitudinally drives the fixation helix 120. As the first portion 128 rides along the guide 182, a distal portion of the fixation helix 120 extends out from a distal portion of the lead.

The lead assembly described above provides several advantages, for example, the ease of manufacturability is increased in that through-put times are reduced, and fewer, less complex parts can be used to manufacture the lead assembly, or to manipulate the lead assembly. Furthermore, the overall size of the lead can be reduced, assisting in minimizing trauma to the patient during implantation of the lead. Despite the reduction in size, the lead assembly maintains an ability to resist drag or jamming of the helix, which was a common shortcoming of conventional devices. Further benefits include the options that are available to modify the device, such as the ability to include single pitch or dual pitch of the helix.

It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1. A lead assembly comprising:
a lead body extending from a distal end to a proximal end;
a conductor disposed within the lead body;
a piston movably disposed within the lead body; and
a fixation helix supported by the piston at a first portion of the fixation helix, the first portion of the fixation helix forming a drive mechanism that advances the fixation helix.

2. The lead assembly as recited in claim 1, wherein the first portion of the fixation helix is coupled with the piston.

3. The lead assembly as recited in claim 1, wherein the piston has a recess, and at least a portion of the first portion of the fixation helix is disposed within the recess.

4. The lead assembly as recited in claim 3, wherein the recess has a helical shape.

5. The lead assembly as recited in claim 3, wherein the recess has a first width and the first width is less than a diameter of the first portion of the fixation helix.

6. The lead assembly as recited in claim 3, wherein approximately ⅓ to ½ of a diameter of the fixation helix is disposed within the recess.

7. The lead assembly as recited in claim 1, further comprising a housing portion disposed near the distal end of the lead body, and a guide is disposed on an inner surface of the housing portion, and the guide guides the drive mechanism.

8. A lead assembly comprising:
a lead body extending from a distal end to a proximal end;
a housing disposed near the distal end of the lead body;
a conductor disposed within the lead body;
a piston movably disposed within the housing;
a fixation helix coupled with the piston along a first longitudinal portion of the fixation helix, the first portion of the fixation helix forming a drive mechanism that advances the fixation helix; and
a guide disposed within an inner surface of the housing.

9. The lead assembly as recited in claim 8, wherein the guide is a helical guide.

10. The lead assembly as recited in claim 8, wherein the helical guide is a segmented helical guide.

11. The lead assembly as recited in claim 8, wherein the fixation helix is coupled with the piston along a recess within the piston.

12. The lead assembly as recited in claim 8, wherein the fixation helix has an inner diameter and the piston has an outer diameter, and the outer diameter is greater than the inner diameter prior to coupling the fixation helix with the piston.

13. The lead assembly as recited in claim 8, wherein the fixation helix is coupled with the piston along a helical recess within the piston.

14. The lead assembly as recited in claim 8, wherein the fixation helix is electrically coupled with the conductor.

15. A lead assembly comprising:
a conductor;
a piston electrically coupled with the conductor; and
an active fixation helix supported by the piston at a first portion of the fixation helix, the first portion of the fixation helix forming a drive mechanism that longitudinally advances and retracts the fixation helix.

16. The lead assembly as recited in claim 15, wherein the active fixation helix is electrically coupled with the piston.

17. The lead assembly as recited in claim 15, wherein the active fixation helix is recessed within a portion of the piston.

18. The lead assembly as recited in claim 15, wherein the active fixation helix is mechanically coupled with the piston.

19. A method comprising:
providing a lead assembly including:
a lead body extending from a distal end to a proximal end;
a conductor disposed within the lead body;
a piston movably disposed within the lead body;
a fixation helix supported by the piston at a first portion of the fixation helix, the first portion of the fixation helix forming a drive mechanism;
rotating the fixation helix; and
longitudinally driving the fixation helix with the drive mechanism.

20. The method as recited in claim 19, further comprising recessing at least a part of the first portion of the fixation helix within the piston.

21. The method as recited in claim 19, further comprising recessing approximately ⅓ to ½ of a diameter of the fixation helix within the piston.

22. The method as recited in claim 19, further comprising recessing at least a part of the first portion of the fixation helix within a helical groove of the piston.

23. The method as recited in claim 19, further comprising coupling the first portion of the fixation helix with the piston.
